(19) United States
(12) Patent Application Publication
(10) Pub. No.: US 2023/0218688 A1
(43) Pub. Date: Jul. 13, 2023

(54) LACTOBACILLUS HELVETICUS ZJUIDS12 FOR TREATING ALCOHOLIC LIVER DISEASE AND APPLICATION THEREOF

(71) Applicants: Zhejiang Chinese Medical University, Hangzhou City (CN); Zhejiang University, Hangzhou City (CN)

(72) Inventors: Songtao LI, Hangzhou City (CN); Daxi REN, Hangzhou City (CN); Qinchao DING, Hangzhou City (CN); Feiwei CAO, Hangzhou City (CN); Xiaobing DOU, Hangzhou City (CN); Qiang HAN, Hangzhou City (CN); Jiaomei LI, Hangzhou City (CN); Bin DING, Hangzhou City (CN); Rui GUO, Hangzhou City (CN); Shanglei LAI, Hangzhou City (CN)

(21) Appl. No.: 17/985,317

(22) Filed: Nov. 11, 2022

(30) Foreign Application Priority Data
Jan. 8, 2022 (CN) 2022100177608

(51) Int. Cl. A61K 35/747; C12N 1/20; A61P 1/16

(52) U.S. Cl. CPC A61K 35/747; C12N 1/205; A61P 1/16

(57) ABSTRACT

Disclosed is Lactobacillus helveticus ZJUIDS12 for treating alcoholic liver disease and application thereof, belonging to food microorganisms. The Lactobacillus helveticus ZJUIDS12 has a strain preservation number of CGMCC NO. 23997. Moreover, disclosed is an application of the Lactobacillus helveticus ZJUIDS12 in preparing products for treating liver injury.

### CROSS-REFERENCE TO RELATED APPLICATIONS

This application claims priority to Chinese Patent Application No. 202210017760.8, filed on Jan. 8, 2022, the entire contents of which are incorporated herein by reference.

### TECHNICAL FIELD

The present application generally relates to food microorganisms, and in particular to Lactobacillus helveticus ZJUIDS12 for treating an alcoholic liver disease and application thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 22049TDFS-USP1-2022-12065-SL.xml. The XML file is 3,275 bytes; was created on Nov. 11, 2022; and is being submitted electronically via EFS-Web.

### BACKGROUND

The liver is a main organ for metabolizing alcohol, and the alcohol is a common pathogenic factor for chronic liver diseases. Research reports that alcoholic liver disease (ALD) has gradually become a chronic liver disease worldwide. In an early stage of ALD, lipids enter liver cells induced by alcohol, gradually accumulate and further form fatty livers. At this time, if patients still drink alcohol, the fatty livers may further develop into hepatic fibrosis or even cirrhosis. At present, ALD is a global concern, but there are no clinically safe and effective medicines for treating ALD. Therefore, how to effectively prevent and treat ALD is an urgent problem in clinical medicine.

Lactobacillus helveticus, an important industrial microbial ferment, is first isolated from western cheeses and mainly used for fermentation of the various cheeses. The Lactobacillus helveticus has an extremely strong proteolytic activity, and its fermented dairy products contain a high content of peptides. Therefore, the Lactobacillus helveticus have a potential to produce bioactive peptides. In addition to cheese making, a growing number of studies show that the Lactobacillus helveticus may promote health.

Chinese Patent No. 201910558554.6 disclosed Lactobacillus helveticus for relieving alcoholic liver injury and application thereof, belonging to a technical field of microorganisms. The Lactobacillus helveticus used in Chinese Patent No. 201910558554.6 is taxonomically named as Lactobacillus helveticus L551, and preserved in China General Microbiological Culture Collection Center (CGMCC) on Apr. 11, 2018, with a strain preservation number of CGMCC NO. 15604. The Lactobacillus helveticus L551 is acid-resistant, has an inhibitory effect on increased alanine aminotransferase (ALT), aspartate aminotransferase (AST) as well as endotoxin in serum, malondialdehyde (MDA) in the liver, and inflammatory factors in liver cells caused by the alcohol, restores activities of glutathione (GSH) and superoxide dismutase (SOD) in the liver, reduces liver fatty blisters and decreases inflammatory cell infiltration. Therefore, the Lactobacillus helveticus L551 may effectively relieve liver injury caused by the alcohol.

### SUMMARY

An objective of the application is to provide Lactobacillus helveticus ZJUIDS12 for treating an alcoholic liver disease and application thereof.

To solve the objective, the application provides Lactobacillus helveticus ZJUIDS12 with a strain preservation number of CGMCC NO. 23997.

Further, a complete sequence of 16S rDNA of the Lactobacillus helveticus ZJUIDS12 is shown in SEQ ID No: 1.

The application also provides an application of the Lactobacillus helveticus ZJUIDS12 in preparing products for treating liver injury.

Further, the products for treating the liver injury include Lactobacillus helveticus ZJUIDS12 powder preparations, heat-killed Lactobacillus helveticus ZJUIDS12, contents and fermentation broth products.

In the application, strain ZJUIDS12 is taxonomically named as the Lactobacillus helveticus, and preserved in CGMCC on Nov. 29, 2021, with the strain preservation number of CGMCC NO. 23997. The CGMCC address is No. 3, Courtyard 1, Beichen West Road, Chaoyang District, Beijing City.

In the application, the Lactobacillus helveticus ZJUIDS12 is extracted from Mongolian traditional fermented food sour cream, and is identified through bacterial morphology, bacterial physiology and culture characteristics combined with 16S rDNA sequencing.

In the application, the Lactobacillus helveticus ZJUIDS12 has significant effects on treating the liver injury, antioxidation in vivo and in vitro, restoring intestinal barrier and improving intestinal flora.

Compared with existing Lactobacillus helveticus, the Lactobacillus helveticus ZJUIDS12 has the following advantages.

Through in vivo animal experiments, it may be found that the strain of the application has strong effects on treating the alcoholic liver disease in vivo, a strong antioxidant capacity and an inhibition ability for Helicobacter pylori; in addition, not only the strain, but also the Lactobacillus helveticus ZJUIDS12 has the significant effects on treating the alcoholic liver disease. Moreover, the Lactobacillus helveticus ZJUIDS12 of the present application also has the antioxidant capacity in vitro, adhesion property (surface hydrophobicity), bile salt resistance, antibiotic susceptibility, and pathogenic bacteria resistance. Furthermore, the heat-killed Lactobacillus helveticus ZJUIDS12 also has the significant effects on treating the alcoholic liver disease.

### BRIEF DESCRIPTION OF THE DRAWINGS

In order to clearly explain the embodiments of the present application or the technical schemes, the following briefly introduces the drawings to be used in the embodiments.

FIG. 1 shows colony morphology of Lactobacillus helveticus ZJUIDS12.

FIG. 2 shows Gram-stained morphology of Lactobacillus helveticus ZJUIDS12.

FIG. 3 shows identification of 16S rDNA of Lactobacillus helveticus ZJUIDS12 by electrophoresis.

FIG. 4 shows effects of Lactobacillus helveticus ZJUIDS12 on ALT and AST in mouse plasma.

FIG. 5 shows effects of Lactobacillus helveticus ZJUIDS12 on free fatty acids (FFA) in mouse plasma.

FIG. 6 shows effects of Lactobacillus helveticus ZJUIDS12 on triglyceride (TG) in a mouse liver.

FIG. 7 shows effects of Lactobacillus helveticus ZJUIDS12 on MDA, total superoxide dismutase (T-SOD) and catalase (CAT) in a mouse liver.

FIG. 8 shows effects of Lactobacillus helveticus ZJUIDS12 on fatty acid synthesis genes (Srebp1c and FAS) in a mouse liver.

FIG. 9 shows effects of Lactobacillus helveticus ZJUIDS12 on tight junction protein genes (ZO-1 and Claudin-1) in mouse intestine.

FIG. 10 shows effects of Lactobacillus helveticus ZJUIDS12 on short-chain fatty acids (SCFAs) (acetic acid, propionic acid and butyric acid) in mouse feces.

FIG. 11 shows effects of Lactobacillus helveticus ZJUIDS12 on probiotics in mouse colon contents.

FIG. 12 shows effects of heat-killed Lactobacillus helveticus ZJUIDS12 on ALT and AST in mouse plasma.

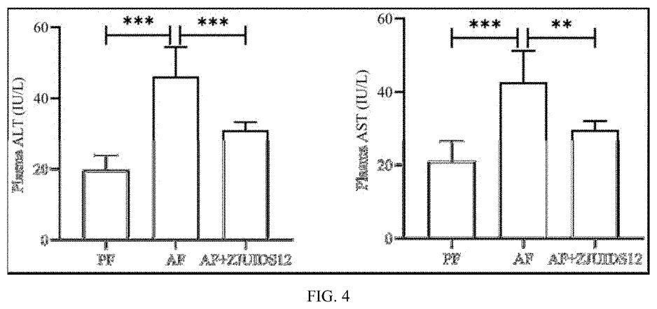

In FIG. 4-FIG. 12, PF represents control groups, AF represents alcohol-treated groups, AF+ZJUIDS12 represents alcohol- and ZUIDS12-treated groups, and AF+ZUIDS12 (heat-killed) represents alcohol- and heat-killed ZUIDS12-treated groups.

### DETAILED DESCRIPTION OF THE EMBODIMENTS

The technical schemes of the present application are clearly and completely described below with reference to the drawings, and it is clear that the described embodiments are a part of the embodiments of the present application, and not all of them.

### Embodiment 1 Extraction and Identification of Lactobacillus helveticus ZJUIDS12

1. Extraction of the Lactobacillus helveticus ZJUIDS12

1.1. Sample Source

A strain ZJUIDS12 used in the application is extracted from sour cream made by herdsmen in Inner Mongolia, and 20 samples are collected.

1.2. Isolation and Purification of the Strain ZJUIDS12

Collecting about 5 gram (g) of the fresh samples with a sterile tube, immediately transporting the samples to a laboratory for isolation: putting 1 g of the samples into a 9 milliliter (mL) of MRS broth medium, evenly mixing by vortex, and performing enrichment culture at 37 degree Celsius (° C.) for 48 hours (h); then, pipetting 1 mL of enriched solution in a super clean bench, diluting with stroke-physiological saline solution (SPSS) in ten-fold gradients, taking 100 microliter (μL) of diluted solution at the dilution gradients of 10⁻⁶, 10⁻⁷ and 10⁻⁸ respectively, spreading them on a MRS agar medium, and culturing them at 37° C. for 48 h; after culturing, selecting a plate with 50-150 single colonies from the MRS agar medium, selecting typical colonies, multiple streaking on a MRS agar plate for purification until the colonies on a whole plate are consistent in morphology, selecting the single colonies to the MRS broth medium for the enrichment culture, and freezing and storing all the obtained strain ZJUIDS12 at −80° C. in the MRS broth medium containing 40 percent (%) of glycerol.

2. Identification of the Lactobacillus helveticus ZJUIDS12

2.1. Colony Characteristics

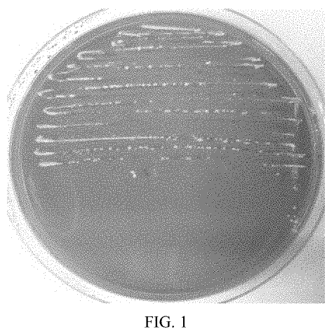

After culturing the isolated and purified Lactobacillus helveticus ZJUIDS12 in the MRS agar medium for 48 h, the Lactobacillus helveticus ZJUIDS12 has a diameter of 0.3-1.5 millimeter (mm), a smooth surface, irregular as well as slightly rough edges, and strong yogurt flavor, as shown in FIG. 1.

2.2. Microscopic Morphology

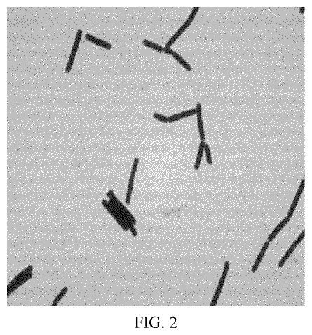

A colony smear of the Lactobacillus helveticus ZJUIDS12: positive after Gram staining, no spores producing, straight bacilli with round ends, single, paired or in short chain-like, as shown in FIG. 2.

2.3. Identification of 16s rDNA

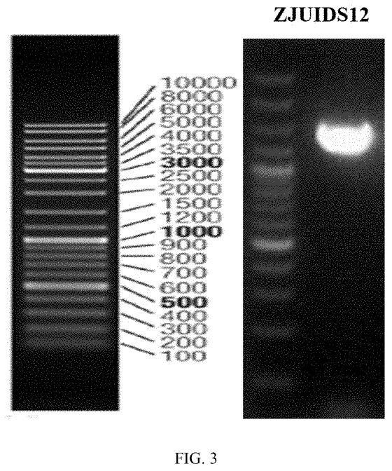

Extracting genomic DNA of target Lactobacillus with an Ezup column type bacterial genomic DNA extraction kit, taking the genomic DNA of the extracted Lactobacillus as a template for polymerase chain reaction (PCR) amplification, using bacterial universal primers 27F and 1492R to perform 16S rDNA PCR experiment, and after the PCR amplification, taking PCR products for detecting and photographing by agarose gel, in which an amplified fragment length is about 1.2 kilobase pairs (kbp), as shown in FIG. 3; sequencing the PCR products by BGI Co., Ltd., in which results are shown as SEQ ID NO.1; and carrying out basic local alignment search tool (BLAST) sequence alignment on National Center of Biotechnology Information (NCBI) website, in which the results show that homology of the sequence and the identified 16S rDNA sequence of the Lactobacillus helveticus ZJUIDS12 exceeds 99%. By combining the results of the sequence alignment of the Lactobacillus ZJUIDS12 with physiological and biochemical results, it is detected that the extracted Lactobacillus ZJUIDS12 is the Lactobacillus helveticus ZJUIDS12.

In the application, the strain ZJUIDS12 is taxonomically named as the Lactobacillus helveticus, and preserved in CGMCC on Nov. 29, 2021, with the strain preservation number of CGMCC NO. 23997, and the CGMCC address is No. 3, Courtyard 1, Beichen West Road, Chaoyang District, Beijing.

In the application, ZJUIDS12 solution is prepared is as follows:

streaking and activating the strain ZJUIDS12 stored in a glycerol tube on the MRS agar plate for 2-3 times, then selecting the single colonies for expanded culture in a MRS liquid medium at 37° C. for 18-24 h, until a concentration of the ZJUIDS12 solution reaches about 10⁹-10¹⁰ colony forming unit per milliliter (CFU/ml).

The ZJUIDS12 solution is used as ZJUIDS12 suspension (as living strain solution). In actual use, the concentration of the ZJUIDS12 solution may be adjusted to a required concentration by using SPSS, such as 5×10⁹ CFU/mL.

The ZJUIDS12 suspension is heat-killed under a high pressure at 121° C. for 30 minutes (min) to obtain heat-killed ZJUIDS12 suspension.

### Embodiment 2 Treatment of an Alcoholic Liver Disease with the Lactobacillus helveticus ZJUIDS12

1. Laboratory animals: 24 C57BL/6 male mice, purchased from Shanghai SLAC Laboratory Animal Co., Ltd. with a company license number of SCXK (Shanghai) 2017-0005, and bred in Zhejiang Chinese Medical University Laboratory Animal Research Center and in a specific pathogen free (SPF) environment.

Reagents: Lieber-DeCarli ethanol liquid diet (product code: TP-4030B; Trophic Animal Feed High-Tech Co., Ltd., China); Lieber-DeCarli control ethanol liquid diet (product code: TP-4030D; Trophic Animal Feed High-Tech Co., Ltd, China); choline and vitamin (Trophic Animal Feed High-Tech Co., Ltd., China); anhydrous ethanol (CAS-NO: 64-17-5, lichrosolv); ALT kits (article number: C009-2; Nanjing Jiancheng Bioengineering Institute); AST kits (article number: C010-2; Nanjing Jiancheng Bioengineering Institute); FFA kits (article number: A042-2-1; Nanjing Jiancheng Bioengineering Institute); TG kits (article number: E1013; Beijing Applygen Technology Co., Ltd.); MDA assay kits (article number: A003-1; Nanjing Jiancheng Bioengineering Institute); T-SOD assay kits (article number: A001-1; Nanjing Jiancheng Bioengineering Institute); and CAT assay kits (article number: A007-1; Nanjing Jiancheng Bioengineering Institute).

2. Method

2.1. Feeding the Laboratory Animals

After a week of adaptive feeding in the SPF animal laboratory, C57BL/6 mice of 8 weeks old are randomly divided into 3 groups, with 8 mice in each group, namely a control ethanol liquid diet+SPSS group (PF group), an ethanol liquid diet+SPSS group (AF group) and an ethanol liquid diet+Lactobacillus helveticus ZJUIDS12 group (AF+ZJUIDS12 group).

The mice in the PF Group are fed with the Lieber-DeCarli ethanol liquid diet for 4 weeks. During the period, an amount of the control ethanol liquid diet in the PF group is adjusted according to intake of the Lieber-DeCarli ethanol liquid diet in the AF group, and each mouse is given a gavage of 0.2 mL of SPSS once a day. AF+ZJUIDS12 group is fed with the Lieber-DeCarli control ethanol liquid diet on days 1-3, with the ethanol liquid diet with 5.5% of calorie radio on days 4-5, with the ethanol liquid diet with 11% of calorie radio on days 6-7, with the ethanol liquid diet with 22% of calorie radio on week 2, with the ethanol liquid diet with 11% of the calorie radio on week 3 and with the ethanol liquid diet with 11% of the calorie radio on week 4. From the first day, each mouse in the AF group is given the gavage of 0.2 mL of SPSS once a day, and each mouse in the AF+ZJUIDS12 group is given the gavage of 0.2 mL of the ZJUIDS12 solution (0.2 mL of the ZJUIDS12 solution contains 10¹ CFU of ZJUIDS12, and the SPSS is used as a solvent).

The liquid diet eaten by the mice is changed into the newly prepared liquid diet every day, weight of the mice is recorded every week, after feeding, the mice are anesthetized by using 1% of pentobarbital intraperitoneal injection, blood samples are taken from inferior vena cava to detect ALT, AST and FFA, and liver and ileum tissues of the mice are taken to detect related indexes.

3. Feed Formula

(1) Preparation Method of the Control Ethanol Liquid Diet for the Mice in the PF Group:

TABLE 1
Formula of liquid feed for the mice in the PF group (1 liter (L))

| Lieber-DeCarli control ethanol liquid diet | Feed ingredients | Dextrin |
|---|---|---|
| (g) | (g) | (g) |
| 224.13 | 106.63 | 117.5 |

Adding about 600 mL of water, stirring and dissolving completely, and then adding the water to a constant volume of 1 L.

Feed ingredients are purchased from Trophic Animal Feed High-Tech Co., Ltd., including casein, L-cystine, DL-methionine, corn oil, olive oil, safflower oil, cellulose, minerals, vitamins, choline tartrate, xanthan gum and Tert-butylhydroquinone (TBHQ).

(2) Preparation Method of the Liquid Feed with Different Ethanol Concentrations

1 L of the liquid feed is taken as an example.

TABLE 2
Formula of the liquid feed (1 L) for the mice in an experimental group

| Calorie radio | Lieber-DeCarli ethanol | Ethanol | Feed | Dextrin |
|---|---|---|---|---|
| of Ethanol | liquid diet (g) | (mL) | ingredients (g) | (g) |
| 0% | 224.13 | 0 | 106.63 | 117.5 |
| 5.5% | 210.38 | 10.47 | 106.63 | 103.75 |
| 11% | 196.63 | 20.94 | 106.63 | 90 |
| 22% | 169.13 | 41.88 | 106.63 | 62.5 |
| 27% | 156.63 | 51.4 | 106.63 | 50 |
| 32% | 144.13 | 60.91 | 106.63 | 37.5 |

Adding about 600 mL of the water, stirring and dissolving completely, and then adding the water to the constant volume of 1 L.

4. Index Detection

Referring to specifications of the kits purchased from Nanjing Jiancheng Bioengineering Institute or Beijing Applygen Technology Co., Ltd., the methods are as follows.

4.1. Detection of Plasma ALT

Directly sampling and detecting the plasma samples of the mice, preheating matrix solution at 37° C. in advance, adding 20 μL of the matrix solution and 5 μL of samples to be detected into a measuring hole, evenly mixing, adding 20 μL of the matrix solution into a control hole, and incubating at 37° C. for 30 min; adding 20 μL of 2,4-dinitrophenylhydrazine solution into the measuring hole and the control hole respectively, adding 5 μL of the samples to be detected into the control hole, evenly mixing, and incubating at 37° C. for 20 min; adding 200 μL of 0.4 mole per liter (mol/L) sodium hydroxide solution into each hole, evenly mixing, letting stand at room temperature for 15 min at a wavelength of 510 nanometer (nm), measuring an optical density (OD) value of each hole with a microplate reader, and obtaining corresponding ALT/Glutamic Pyruvic Transaminase (GPT) activity unit according to standard curve.

4.2. Detection of Plasma AST

Directly sampling and detecting the plasma samples of the mice, preheating the matrix solution at 37° C. in advance, adding 20 μL of the matrix solution and 5 μL of the samples to be detected into the measuring hole, evenly mixing, adding 20 μL of the matrix solution into the control hole, and incubating at 37° C. for 30 min; adding 20 μL of 2,4-dinitrophenylhydrazine solution into the measuring hole and the control hole respectively, adding 5 μL of the samples to be measured into the control hole, evenly mixing, and incubating at 37° C. for 20 min; adding 200 μL of the 0.4 mol/L sodium hydroxide solution into each hole, evenly mixing, standing at room temperature for 15 min at the wavelength of 510 nm, measuring the OD value of each hole with the microplate reader, and obtaining the corresponding ALT/GPT activity unit according to the standard curve.

4.3. Detection of Plasma FFA

Adding 4 μL of double distilled water into a blank hole, 4 μL of standard substance into a calibration hole, and 4 μL of the samples into a sample hole, and then adding 200 μL of a reagent I into the three holes; evenly mixing, incubating at 37° C. for 5 min, reading an absorbance value A1, adding 50 μL of a reagent II into the three holes, evenly mixing, incubating at 37° C. for 5 min, reading an absorbance value A2, calculating a value of A2−A1, and carrying out two-point calibration calculation.

4.4. Detection of Liver TG

Accurately weighing 100 milligram (mg) of the liver, adding lysis solution according to a proportion of the lysis solution (μL):the liver (mg)=20:1, grinding the tissues by using a full-automatic rapid grinding instrument, waiting for 10 min, taking a proper amount of supernatant, transferring the supernatant into a 1.5 mL centrifuge tube, and performing protein quantification on the remaining lysis solution by using a bicinchonininc acid (BCA) method; heating the supernatant at 70° C. in a metal bath for 10 min, centrifuging the supernatant at 2000 revolutions per minute (rpm) at room temperature for 5 min, taking the supernatant for TG detection, taking 10 μL of the supernatant in a 96-well plate, diluting 4 mM of glycerol standard substance in multiple proportions of 1000, 500, 250, 125, 62.5, 31.25, 15.625 and 7.8125 μmol/L, taking 10 μL of each dilution in the 96-well plate, preparing a working solution according to the specification of the kit purchased from Beijing Applygen Technology Co., Ltd., adding 190 μL of the working solution into samples, incubating at 37° C. for 15 min, and measuring the OD value by using the wavelength of 550 nm.

4.5. Detection of MDA

Accurately weighing liver tissues, adding 9 times of SPSS according to the proportion of liver tissues weight (g):SPSS volume (mL)=1:9, shearing the liver tissues, preparing homogenate in ice water bath, centrifuging at 3000 revolutions per minute (r/min) for 10 min, and taking 10% of liver homogenate supernatant to be detected; adding 0.1 mL of anhydrous ethanol into a blank tube, adding 0.1 mL of 10 nanomole (nmol)/mL standard substance into a reference tube, adding 0.1 mL of samples to be detected into a detection tube and a control tube, adding 0.1 mL of reagent I into the four tubes respectively, evenly mixing, adding 3 mL of reagent II application solution into the four tubes, adding 1 mL of reagent III application solution into the blank tube, the reference tube and the detection tube, adding 1 mL of 50% glacial acetic acid into the control tube, evenly mixing, incubating at 95° C. for 40 min, taking out, cooling, centrifuging at 3500-4000 r/min for 10 min, taking out supernatant, detecting the OD value at the wavelength of 532 nm, and calculating MDA content in each group according to a calculation formula.

4.6. Detection of T-SOD

Taking 10% of the liver homogenate supernatant to be detected, adding 1 mL of the reagent I into two tubes, adding 0.05 mL of samples into the detection tube, adding 0.05 mL of distilled water into the control tube, adding 0.1 mL of the reagent II application solution, the reagent III application solution and reagent IV application solution into the two tubes respectively, evenly mixing with a vortex mixer, incubating at 37° C. for 40 min, adding 2 mL of a color developing agent into the two tubes respectively, evenly mixing, placing at room temperature for 10 min, and measuring the OD value at the wavelength of 550 nm.

4.7. Detection of CAT

Taking a proper amount of 10% liver homogenate, diluting the 10% liver homogenate with SPSS to prepare 1% liver homogenate according to the proportion of liver homogenate:SPSS=1:9, detecting a protein concentration of the 1% liver homogenate by using a BCA kit, preparing a substrate solution with the absorbance value between 0.5 and 0.55, and pre-heating the substrate solution to 25° C. for later use; taking a quartz cuvette with an optical path of 1 centimeter (cm), using ultraviolet light of 240 nm, zeroing with the double distilled water for later use; adding 0.02 mL of pretreated samples into a bottom of the quartz cuvette, quickly transferring 3 mL of the substrate solution preheated to 25° C. with the OD value between 0.5 and 0.55 into the quartz cuvette by using a 5 mL pipette, measuring the absorbance value at the wavelength of 240 nm, recording an OD1 value, measuring the absorbance value once again at 1 min without taking out the quartz cuvette, recording an OD2 value, and calculating CAT activity in each group according to a calculation formula.

4.8. Real-Time Quantitative Polymerase Chain Reaction (Real-Time PCR)

(1) Taking out the tissues from a refrigerator at −80° C. to an ice box, weighing about 0.02 g of the tissues in an eppendorf tube (EP tube) by an electronic balance, and precooling a centrifuge at 4° C.;

(2) adding 1 mL of Trizol and 3 steel balls into the EP tube filled with the tissues, grinding by a grinder, taking out liquid and the steel balls, and carrying out a reaction at room temperature for 10 min;

(3) adding 200 μL of chloroform into the EP tube, violently shaking for 30 seconds (s) for evenly mixing, placing the EP tube on ice, and letting the EP tube stand for 5-10 min;

(4) placing the EP tube in the centrifuge at 4° C., and centrifuging at 12000 rpm for 15 min;

(5) pipetting aqueous phase (supernatant) from the centrifuged samples into a 1.5 mL new centrifuge tube;

(6) adding isopropanol solution with the same volume as the centrifuged and extracted solution, slightly reversing for evenly mixing, and letting samples stand at −20° C. for 20 min;

(7) taking out the samples, and centrifuging the samples at 4° C. and 12000 rpm for 15 min;

(8) discarding the supernatant to obtain white (or colorless and transparent) precipitate after centrifuging, adding 100-300 μL of precooled 75% ethanol prepared with diethypyrocarbonate (DEPC) water along the inner wall of the centrifuge tube, and washing for 2-3 times;

(9) discarding liquid, air drying at room temperature for about 15 min, adding 20-50 μL of the precooled DEPC water into the centrifuge tube to dissolve the air-dried precipitate (RNA) at the bottom of the centrifuge tube, and storing in the refrigerator at −20° C. for later use;

(10) detecting an RNA concentration with an ultramicro ultraviolet-visible (UV) spectrophotometer, recording results and calculating a sample volume for each group;

(11) as shown in table 1, adding corresponding reaction solution in a reverse transcription kit for premixing, adding samples for reverse transcription, shaking and centrifuging, placing the samples into a PCR Amplifier, setting corresponding PCR reaction conditions in the reverse transcription kit, and storing cDNA samples reversibly transcribed by a reverse transcription instrument in the refrigerator at −20° C. for later use;

(13) adding reactants into 0.2 mL of PCR eight-connected tubes, amplifying a target gene by a fluorescent quantitative PCR amplifier by using cDNA as a template, and detecting the fluorescent quantitative PCR;

(14) shaking the mixed reactants for evenly mixing, centrifuging, placing the eight-connected tubes into a quantitative polymerase chain reaction (qRT-PCR) reactor, setting PCR reaction programs as follows: pre-denaturalizing at 94° C. for 5 min; denaturalizing at 94° C. for 30 s, denaturalizing at 60° C. for 30 s and denaturalizing at 72° C. for 30 s, in which the above process is repeated 40 times; extending at 72° C. for 5 min; and preserving at 4° C.; setting a temperature of a hot lid of the PCR amplifier at 105° C.; and

(15) calculating expression change of the target gene by a 2-Quantitation-Comparative CT (ΔΔCT) method.

4.9. Detection of SCFAs

Squeezing and segmenting colon sections with a sterile forcep, taking out colon contents, storing in a cryopreservation tube at −80° C., diluting the colon contents with ultrapure water by 5 times, carrying out the vortex for 3 min, letting suspension stand for 5 min, then centrifuging at 4° C. and 5000×g for 20 min, mixing 1 mL of supernatant with 20 μL of chromatographic-grade phosphoric acid, and injecting the mixture into a chromatographic bottle through a 0.45 micrometer (μm) membrane filter for gas chromatography, in which the gas chromatograph used is GC-2010, which consists of an AOC-20S automatic sampler and a flame ionization detector, with nitrogen as carrier gas and a 3 mL/min flow rate; installing a high polarity column of SH-stable wax on the gas chromatograph, in which sample size is 0.2 μL, a split injection ratio is 50 and an injection temperature is 200° C.; injecting ethyl acetate in each sample as a blank solvent to eliminate any memory effect; setting an initial column temperature at 80° C. and keeping for 1 min, increasing the temperature to 170° C. at a rate of 8° C./min, and then increasing to 220° C. at the rate of 20° C./min and keeping for 4 min, which takes a total of 18.75 min; finally, calibrating the SCFAs contents with an external standard method according to standard curve of the SCFAs.

4.10. 16S Ribosomal RNA (16s rRNA) Gene Sequencing

The collected colon contents are sent to Hangzhou MKBio Company for total DNA isolation and 16s rRNA high-throughput sequencing. The 16s rRNA is amplified in V3-V4 region, amplicon is purified by QIA quick PCR purification kit and sequenced on IlluminaNovaseq platform, and an original sequence is quality-controlled by UPARSE. An operational taxonomic unit (OTU) is constructed by binding sequence to clusters with sequence similarity greater than 97% by using QIIME.

5. Experimental Results

It may be seen from FIG. 4 that ZJUIDS12 significantly reduces increased ALT and AST contents caused by alcohol intake. ALT mainly exists in cytoplasm of liver cells, an intracellular concentration of ALT is 1000-3000 times higher than the concentration of the ALT in serum, and as long as 1% of the liver cells are destroyed, serum enzyme level is doubled. Therefore, ALT is evaluated by the World Health Organization as the most sensitive substance for liver damage. Lower the ALT content means lower degree of the liver damage. In addition, AST mainly exists in mitochondria of the liver cells, and is also one of the sensitive substances for the liver damage.

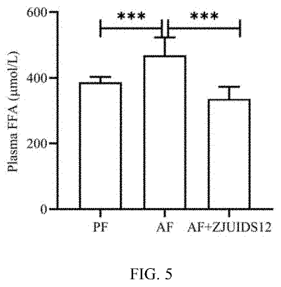

It may be seen from FIG. 5 that ZJUIDS12 significantly reduces increased FFA content caused by the alcohol intake. FFA is a decomposition product of TG. Normally, the FFA content in the plasma is very low, and with increase of the FFA content, permeability of mucosa is changed, leading to damage of the mucosa. Moreover, if the liver takes in too much FFA to be completely oxidized by the mitochondria of the liver cells, it leads to the increase of TG and further form fatty livers.

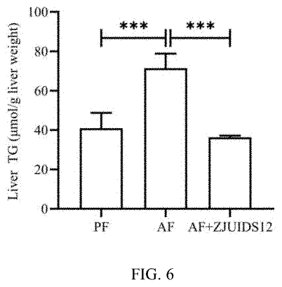

It may be seen from FIG. 6 that ZJUIDS12 significantly reduces increased liver TG contents of caused by the alcohol intake. Compared with the PF group, the liver TG content of the mice in the AF group is significantly increased, while that in AF+ZJUIDS12 group is significantly decreased. Therefore, it is concluded that ZJUIDS12 may effectively reduce the liver TG content of the mice.

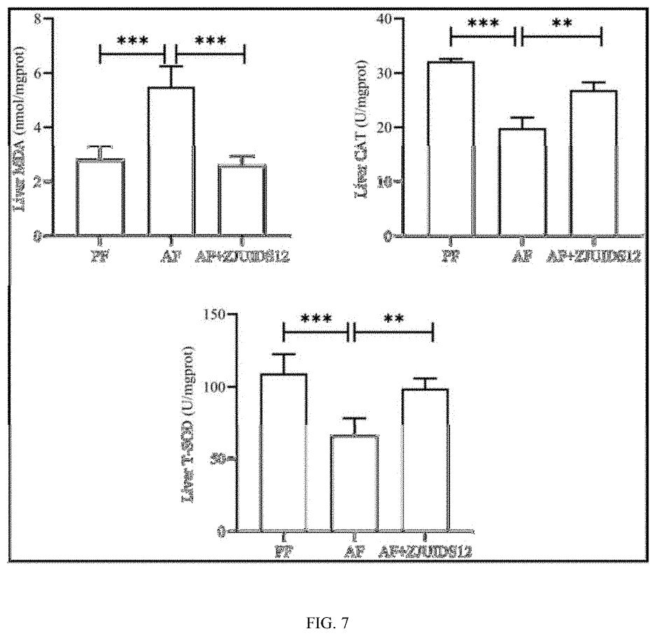

It may be seen from FIG. 7 that ZJUIDS12 has strong antioxidant capacity. Normally, antioxidant enzymes in a natural antioxidant defense system of organisms may cooperate with antioxidants in feeds or medicines to remove peroxides. Superoxide dismutase (SOD) and CAT are the most important antioxidant enzymes. SOD disproportionates superoxide anion into hydrogen peroxide, while CAT may reduce the hydrogen peroxide, thus preventing production of highly toxic hydroxyl radicals. A degree of free radical attack on cells is indirectly judged by detecting a level of MDA, a lipid peroxidation product.

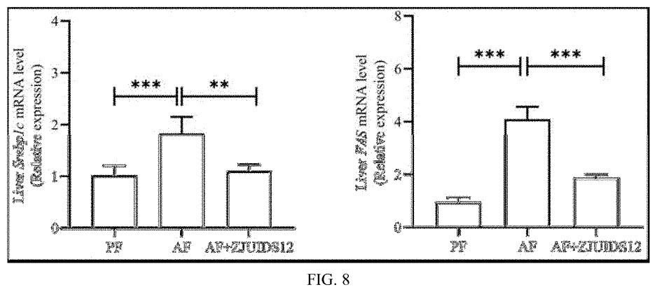

It may be seen from FIG. 8 that ZJUIDS12 may inhibit de novo synthesis of the liver TG. Sterol-regulatory element binding proteins (SREBP-1c) are main transcription factors regulating genes related to synthesis of liver fatty acid and the liver TG, and is closely related to lipotoxicity caused by excessive accumulation of the liver TG in the liver cells. FAS is a target gene of SREBP-1c. In FIG. 8, expression of SREBP-1c and FAS in the AF group is significantly increased, while ZJUIDS12 may significantly inhibit gene expression, which indicates that ZJUIDS12 may inhibit the de novo synthesis of the liver TG.

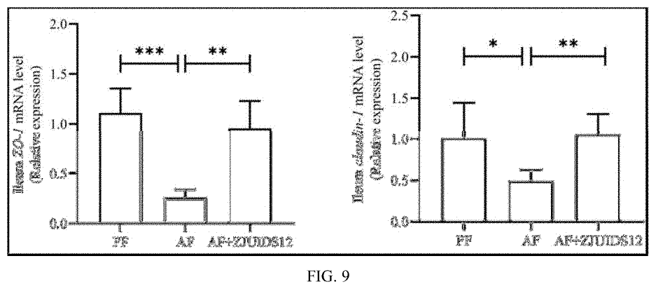

It may be seen from FIG. 9 that ZJUIDS12 may improve intestinal mucosal barrier. Tight junction is a main connection mode between intestinal epithelial cells and plays an important role in protecting mechanical barrier and improving permeability of intestinal mucosal epithelium. Moreover, tight junction proteins are important protein molecules that form the intestinal mucosal barrier and influence permeability of intestinal wall, and have great influence on composition and functions of the tight junction, in which ZO-1 and Claudin 1 are important factors forming the intercellular tight junction, and ZJUIDS12 may significantly improve decreased gene expression of ZO-1 and Claudin 1 caused by the alcohol intake.

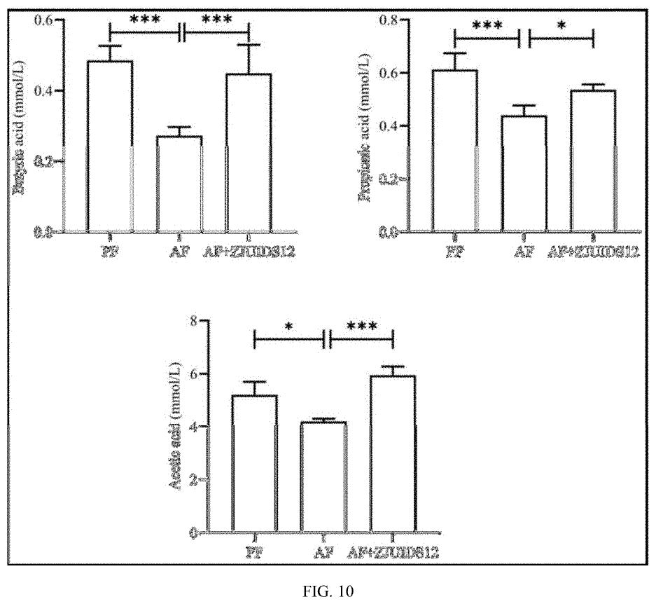

It may be seen from FIG. 10 that ZJUIDS12 may promote synthesis of the intestinal SCFAs. SCFAs, saturated fatty acids with 1-6 carbon atoms, are main metabolites produced by intestinal microbial fermentation and maintains a redox equivalent in an intestinal aerobic environment. Moreover, 95% of SCFAs are acetic acid (C2), propionic acid (C3) and butyric acid (C4). ZJUIDS12 may significantly increase C2, C3, and C4 contents in intestinal tract.

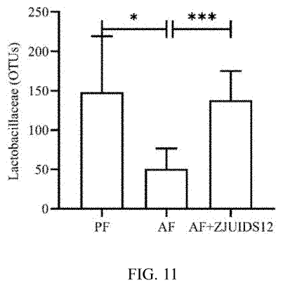

It may be seen from FIG. 11 that ZJUIDS12 may improve intestinal flora. In FIG. 11, a Lactobacillaceae content in the intestinal tract in the AF+ZJUIDS12 group is significantly higher than that in the AF group, and is similar to that in the PF group. Therefore, it shows that ZJUIDS12 may regulate the intestinal flora and improve intestinal health.

### Embodiment 3 Treatment of the Alcoholic Liver Disease with the Heat-Killed Lactobacillus helveticus ZJUIDS12

1. Laboratory animals: 32 C57BL/6 male mice, purchased from Shanghai SLAC Laboratory Animal Co., Ltd. with the company license number of SCXK (Shanghai) 2017-0005, and bred in Zhejiang Chinese Medical University Laboratory Animal Research Center and in the SPF environment.

Reagents: the Lieber-DeCarli ethanol liquid diet (product code: TP-4030B; Trophic Animal Feed High-Tech Co., Ltd., China); the Lieber-DeCarli control ethanol liquid diet (product code: TP-4030D; Trophic Animal Feed High-Tech Co., Ltd, China); the choline and the vitamin (Trophic Animal Feed High-Tech Co., Ltd., China); the anhydrous ethanol (CAS-NO: 64-17-5, lichrosolv); the ALT kit (article number: C009-2; Nanjing Jiancheng Bioengineering Institute); the AST kit (article number: C010-2; Nanjing Jiancheng Bioengineering Institute); and the FFA kit (article number: A042-2-1; Nanjing Jiancheng Bioengineering Institute).

2. Method

2.1. Experimental Grouping

The 32 male mice are divides into 4 groups: 1) PF group: SPSS group; 2) AF group: ethanol liquid diet group; 3) AF+ZUIDS12 group: ethanol liquid diet+Lactobacillus helveticus ZUIDS12 solution group, in which the Lactobacillus helveticus ZUIDS12 solution contains 10⁹ CFU/ml live Lactobacillus helveticus ZUIDS12; 4) AF+ZUIDS12(heat-killed) group: control ethanol liquid diet+heat-killed Lactobacillus helveticus ZUIDS12 solution group, in which the 10⁹ CFU/ml Lactobacillus helveticus ZUIDS12 solution is heat-killed at 121° C. for 30 min.

2.2. Feeding the Laboratory Animals (Referring to Embodiment 2)

After a week of the adaptive feeding in the SPF animal laboratory, the C57BL/6 mice of 8 weeks old are randomly divided into 4 groups, with 8 mice in each group, namely the control ethanol liquid diet+SPSS group (PF group), the ethanol liquid diet+SPSS group (AF group), the ethanol liquid diet+Lactobacillus helveticus ZJUIDS12 group (AF+ZJUIDS12 group) and the ethanol liquid diet+heat-killed Lactobacillus helveticus ZJUIDS12 group (AF+ZJUIDS12(heat-killed) group). Each mouse in the PF group is given the gavage of 0.2 mL of SPSS once a day, each mouse in the AF+ZJUIDS12 group is given the gavage of 0.2 mL of the Lactobacillus helveticus ZJUIDS12 solution, and each mouse in the AF+ZJUIDS12(heat-killed) group is given the gavage of 0.2 mL of the heat-killed Lactobacillus helveticus ZJUIDS12 solution.

3. Experimental Results (Referring to Embodiment 2 for Animal Experiment Method and Detection Indexes)

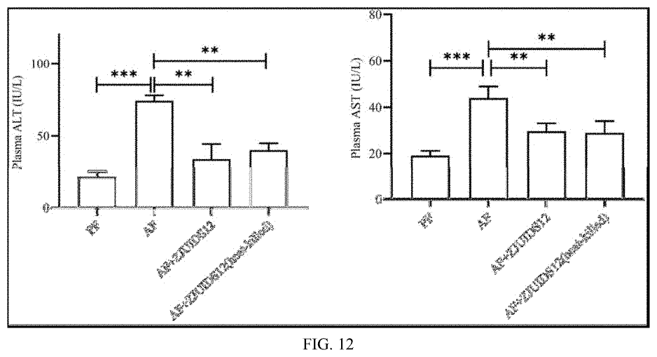

It may be seen from FIG. 12 that not only the Lactobacillus helveticus ZJUIDS12 may treat the alcoholic liver disease, but also the heat-killed Lactobacillus helveticus ZJUIDS12 may reduce plasma ALT and AST levels, which shows that the Lactobacillus helveticus ZJUIDS12 may treat the alcoholic liver disease. Therefore, the heat-killed Lactobacillus helveticus ZJUIDS12 may be used as postbiotics for treating the alcoholic liver disease.

According to the above method of the present application, the Lactobacillus helveticus L551 solution is heat-killed under the high pressure at 121° C. for 30 min.

The ALT and AST levels in a heat-killed Lactobacillus helveticus L551 group are close to those in the AF group, which indicates that the heat-killed Lactobacillus helveticus L551 has no significant effect on treating the fatty livers.

The ALT and AST levels in the AF+ZJUIDS12 (heat-killed) group are similar to those in the AF+ZJUIDS12 group, and are significantly lower than those in the AF group, as shown in FIG. 12.

Therefore, the Lactobacillus helveticus ZJUIDS12 of the present application has a good effect on treating the fatty livers even after being heat-killed, and thus is obviously superior to the Lactobacillus helveticus L551.

### Embodiment 4 Detection of the Antioxidant Capacity of the Lactobacillus helveticus ZJUIDS12

1. Total Antioxidant Capacity (Fluorescence Recovery after Photobleaching (FRAP) Method)

The total antioxidant capacity is detected by a method of Giuberti et al. with some modifications: adding 150 μL of a tripyridyl-triazine (TPTZ) working solution (0.3 M of acetic acid-sodium acetate buffer solution, 20 mM of ferric chloride solution and 10 mM of TPTZ buffer solution, mixed with a proportion of V:V:V=10:1:1, ready-to-use) and 20 μL of samples into an enzyme-linked immuno sorbent assay (ELISA) Plate, shaking for evenly mixing, carrying out a reaction at 37° C. for 10 min, measuring the absorbance value of solution at 593 nm, and substituting the measured absorbance value of the samples into standard curve of ferrous sulfate. The antioxidant capacity of the samples is expressed as a ferrous sulfate equivalent (mol FeSO₄/mL samples). Each sample is repeatedly measured for 3 times for obtaining an average value.

The standard curve of the ferrous sulfate is measured as follows: preparing ferrous sulfate solution with different mass concentrations (0 μRM, 50 μM, 100 μM, 200 μM, 400 μM, 600 μM, 800 μM), mixing the ferrous sulfate solution with the different molar concentrations, 10 mM of the TPTZ buffer solution and 0.3 M of the acetate buffer solution with the proportion of V:V:V=1:1:10, and adding 170 μL of mixed solution to the ELISA plate, carrying out a reaction at 37° C. for 10 min, measuring the absorbance value of the mixed solution at 593 nm, drawing the standard curve with the absorbance value as ordinates and the mass concentrations of the ferrous sulfate as abscissae and measuring.

2. Reducibility

The reducibility is measured by a method of Lin et al. with some modifications: putting 1 mL of samples into a centrifuge tube, adding 1 mL of 0.2 M phosphate buffered saline (PBS) buffer solution with pH 6.6 and 1 mL 1% (w/v) potassium ferricyanide solution, evenly mixing, carrying out water bath at 50° C. for 20 min, cooling in the ice bath, adding 1 mL of 10% trichloroacetic acid, centrifuging at 6000 r/min for 5 min, taking 1 mL of supernatant, adding 1 mL of 0.1% (w/v) ferric trichloride and 1 mL of the distilled water, evenly mixing, waiting for 10 min, and measuring the absorbance value at 700 nm; using the PBS buffer solution or the MRS broth medium instead of the samples as a blank group, in which each sample is repeatedly measured for 3 times for obtaining the average value.

Reducibility (%)=[(As−Ab)/Ab]*100,

where As is an absorbance value of a sample group and Ab is the absorbance value of the blank group.

3. 1,1-Diphenyl-2-Picrylhydrazyl Radical (DPPH) Free Radical Scavenging Ability

The DPPH free radical scavenging ability is measured by a method of Shimada et al. with some modifications: preparing 1000 mg/mL of VC standard solution, diluting the VC standard solution into different concentration gradients (0-30 μg/ml), adding 100 μL of samples to be detected (or the VC standard solution) and 100 μL of 0.2 mM DPPH ethanol solution (prepared by the absolute ethanol, stored at 4° C. in dark, ready-to-use) into the ELISA plate, evenly shaking, then keeping away from light for 30 min at room temperature, and measuring the absorbance values at 593 nm; using 100 μL of the absolute ethanol instead of 100 μL of DPPH ethanol solution as the blank group; using 100 μL of the PBS buffer solution (0.2 M PBS or MRS broth medium with pH 6.6) instead of 100 μL of the samples to be detected as the control group, and blank-zeroing mixed solution of 100 μL of the PBS buffer solution (or the MRS broth medium) and the absolute ethanol. Each sample is repeatedly measured for 3 times for obtaining the average value.

DPPH free radical scavenging ability (%)=[1−(As−Ab)/Ac]*100,

where As is the absorbance value of the sample group, Ab is the absorbance value of the blank group, and Ac is the absorbance value of the control group.

Results are shown in Table 3

TABLE 3
Antioxidant capacity of the Lactobacillus helveticus ZJUIDS12

| | Suspension | | Fermentation supernatant | |
|---|---|---|---|---|
| | ZJUIDS12 | ATCC53103 | ZJUIDS12 | ATCC53103 |
| Total | 78.6 ± 27.7 | 241.1 ± 30.9 | 558.4 ± 5.9* | 440.4 ± 58.7 |
| antioxidant | | | | |
| capacity | | | | |
| Reducibility | 0.09 ± 0.02 | 0.08 ± 0.004 | 0.89 ± 0.02* | 0.76 ± 0.01 |
| DPPH free | 33.5 ± 3.09* | 26.4 ± 1.54 | 87.1 ± 9.26* | 74.1 ± 9.26 |
| radical | | | | |
| scavenging | | | | |
| rate (%) | | | | |

*means significant difference, P < 0.05; **means significant difference, P < 0.01.

As shown in table 3, the total antioxidant capacity, the reducibility and the DPPH free radical scavenging rate of fermentation supernatant of the Lactobacillus helveticus ZJUIDS12 are significantly higher than those of standard strain ATCC53103, thus indicating that the fermentation supernatant and the suspension of the Lactobacillus helveticus ZJUIDS12 have strong antioxidant capacity.

### Embodiment 5 Detection of Inhibition Ability of the Lactobacillus helveticus ZJUIDS12 to Helicobacter pylori

1. Cultivation of the Helicobacter pylori ATCC43504

Coating 100 μL of the Helicobacter pylori on a Columbia blood agar culture medium for activation culture under conditions of a microaerophilic environment (7% of oxygen, 10% of carbon dioxide and 83% of nitrogen) at 37° C. for 72-96 h, activating one generation in a solid culture medium, selecting single colonies, streaking for passage for three generations, selecting purified single colonies and inoculating the purified single colonies into a liquid culture medium of the Helicobacter pylori, in which liquid culture conditions are the same as the solid culture conditions, centrifuging liquid culture mixture at 8000 rpm for 15 min at 4° C., collecting Helicobacter pylori supernatant and precipitate respectively, washing the Helicobacter pylori precipitate with the clean liquid culture medium of the Helicobacter pylori twice, and resuspending to make viable count reach 10⁷ CFU/mL.

2. Cultivation of Experimental Strains

Taking the isolated and purified Lactobacillus helveticus ZJUIDS12 out of the refrigerator at −80° C., unfreezing the Lactobacillus helveticus ZJUIDS12 at room temperature, selecting a small amount of the Lactobacillus helveticus ZJUIDS12 solution in the glycerol tube to activate on a MRS solid culture medium, selecting Lactobacillus single colonies on the MRS solid culture medium after 24 h, streaking the Lactobacillus single colonies for 3 times of passage, inoculating the last activated single colonies in a MRS liquid culture medium, carrying out a static cultivation at 37° C. for 24 h, taking out, centrifuging at 8000 rpm for 15 min at 4° C., collecting supernatant and precipitate respectively, sterilizing the supernatant with a 0.22 μm filter, and washing the precipitate with sterile PBS twice and resuspending, so that the number of the viable count reaches 10⁸ CFU/mL.

3. Growth Inhibition Experiment of the Lactobacillus helveticus ZJUIDS12 on the Helicobacter pylori

An agar diffusion method is used to detect an inhibition activity of the Lactobacillus helveticus ZJUIDS12 extracted in this experiment to the Helicobacter pylori: preparing the Columbia blood agar culture medium, pouring the Columbia blood agar culture medium into petri dishes which are placed with sterile Oxford cups in advance when the Columbia blood agar culture medium is cooled to about 55° C., in which each petri dish is quantitatively added with 15 mL of the Columbia blood agar culture medium, and gently pulling out the Oxford cups with a sterile tweezer after the Columbia blood agar culture medium is cooled.

Taking 100 μL of Helicobacter pylori suspension and evenly spreading the Helicobacter pylori suspension on a Columbia blood agar plate without antibiotics, adding 100 μL of the liquid to be detected into each well, putting the Columbia blood agar plate with fermentation broth in a microaerobic environment at 37° C. for 72-96 h, and measuring a diameter of an inhibition zone with a vernier caliper after the cultivation.

4. Detection of the Inhibition Ability of the Lactobacillus helveticus ZJUIDS12 to Urease Activity

Mixing 40 μL of the Helicobacter pylori suspension with 10 μL of the fermentation supernatant/Lactobacillus suspension of the Lactobacillus helveticus ZJUIDS12, respectively, using 10 μL of a sterile Helicobacter pylori liquid culture medium as the blank control, pipetting 50 μL of the mixed solution, adding the mixed solution into a clean and sterile 96-well plate, culturing the mixed solution at 37° C. in a microaerobic environment for 48 h, taking out the cultured mixed solution, adding 150 μL of urease reagent into each well, observing color change and measuring a OD550 value.

A formula of urease indicator is: 0.9% NaCl, 20 mmol/L urea, 14 μg/mL phenol red, and pH is adjusted to 6.8 with HCl.

Inhibition rates of the Lactobacillus helveticus to the Helicobacter pylori and the urease activity are shown in table 4 below. From table 4, it may be seen that the Lactobacillus helveticus ZJUIDS12 and the fermentation supernatant all have the high inhibition rates to the Helicobacter pylori and the urease activity.

TABLE 4
Detection results of bile salt hydrolase of the Lactobacillus helveticus
ZJUIDS12

| | Inhibition rates to | | Inhibition rates to urease | |
|---|---|---|---|---|
| | Helicobacter pylori (%) | | activity (OD550) | |
| | Fermentation | | Fermentation | |
| Strain | supernatant | Suspension | supernatant | Suspension |
| ZJUIDS12 | 76.52 ± 4.33** | 85.01 ± 1.33** | 0.97 ± 0.00** | 0.37 ± 0.05* |
| Negative | 31.97 ± 1.88 | 31.97 ± 1.88 | 0.39 ± 0.00 | 0.33 ± 0.02 |
| control | | | | |
| Positive | 100 | 100 | — | — |
| control | | | | |

Note:
negative control is common Lactobacillus helveticus; positive control is 0.05 mg/mL metronidazole solution; — is the unmeasured group; average values in the same column marked with * indicate the significant difference (*P < 0.05; **P < 0.01).

### Embodiment 6 Detection of Bile Salt Hydrolase Activity of the Lactobacillus helveticus ZJUIDS12

1. Qualitative Detection of the Bile Salt Hydrolase Produced by the Lactobacillus helveticus ZJUIDS12

Adding 0.3% (m/v, 3 g/1000 ml) sodium deoxytaurocholate, 0.2% (m/v) sodium thioglycolate and 0.37 g/L CaCl₂ into the newly prepared MRS agar culture medium, completely dissolving them, sterilizing them at 121° C. for 15 min, pouring into a sterile plate, placing a piece of sterile filter paper into the sterile plate after solidification, adding 10 μL of the Lactobacillus helveticus ZJUIDS12 suspension (about 10⁸ CFU/mL) to the filter paper, and adding 10 μL of sterile phosphate buffer solution as the blank control; incubating the sterile plate in an anaerobic jar (OXOID) at 37° C. for 72 h. If there is white precipitate around the filter paper, it is considered that the Lactobacillus helveticus ZJUIDS12 has the bile salt hydrolase activity.

Among them, the ZJUIDS12 obtained in embodiment 2 is resuspended by the 0.2 M PBS buffer solution with pH 7.0 to prepare the about 10⁸ CFU/mL ZJUIDS12 suspension.

2. Quantitative Detection of the Bile Salt Hydrolase Activity of the Lactobacillus helveticus ZJUIDS12

Preparation of the Lactobacillus helveticus ZJUIDS12 and the Suspension Refers to Embodiment 2:

Taking 0.1 mL of the supernatant of the Lactobacillus helveticus ZJUIDS12, adding 1.8 mL of 0.1 mol/L PBS buffer solution (0.2 M, pH 7.0) and 0.1 mL of 6 mmol/L sodium taurocholate, culturing mixture at 37° C. for 30 min, then adding 0.5 ml of 15% trichloroacetic acid to stop an enzymatic reaction, and centrifuging; adding 1 mL of ninhydrin chromogenic solution into 0.5 mL of the supernatant, evenly mixing them by the vortex, boiling for 35 min, and measuring the absorbance value at 570 nm after cooling.

One bile salt hydrolase activity unit is defined as an amount of enzymes required to release 1 μmoL of taurine from substrate per minute.

The bile salt hydrolase activity is detected by taking Lactobacillus rhamnosus ATCC53103 as a positive control, and protein concentrations are detected by using bovine serum albumin as standard. All experiments are repeated 3 times.

Table 5 shows quantitative detection results of the bile salt hydrolase activity of the Lactobacillus helveticus ZJUIDS12 and the Lactobacillus rhamnosus ATCC53103. It may be seen from table 5 that the bile salt hydrolase activity of the Lactobacillus helveticus ZJUIDS12 and the Lactobacillus rhamnosus ATCC53103 is above 1.0 U/mg, and the bile salt hydrolase activity of the Lactobacillus helveticus ZJUIDS12 is higher than that of the Lactobacillus rhamnosus ATCC53103.

TABLE 5
Detection results of the bile salt hydrolase of strains

| | | Bile salt hydrolase activity |
|---|---|---|
| Strain | Qualitative analysis | (U/mg) |
| Lactobacillus helveticus | + | 1.55 ± 0.04 |
| ZJUIDS12 | | |
| Lactobacillus rhamnosus | + | 1.10 ± 0.07 |
| ATCC53103 | | |

Note:
+ means precipitation circle, − means no precipitation circle.

The bile salt hydrolase may hydrolyze conjugated bile salt in vivo into free bile salt, but the free bile salt does not participate in enterohepatic circulation and is excreted with feces, so the bile salt hydrolase activity is a key factor to reduce a blood sugar level in vivo. Moreover, the Lactobacillus helveticus ZJUIDS12 provided by the application has the high bile salt hydrolase activity.

### Embodiment 7 Detection of Acid Resistance and Bile Salt Resistance of Lactobacillus helveticus ZJUIDS12

1. Acid Resistance Experiment

Selecting the single colonies of the Lactobacillus helveticus ZJUIDS12, performing expanded culture in the MRS liquid culture medium at 37° C. for 18 h, inoculating 1% of the expanded suspension of the Lactobacillus helveticus ZJUIDS12 into the MRS liquid culture medium, performing culture at 37° C. for 18 h, centrifuging culture solution at 8000 r/min for 5 min at 4° C., collecting the Lactobacillus helveticus ZJUIDS12, and washing with the 0.1 mol/L PBS buffer solution with pH 6.8 for 2 times; suspending the Lactobacillus helveticus ZJUIDS12 in the MRS liquid culture medium with pH 3.0, adjusting initial viable count to about 10⁸ CFU/mL, culturing at 37° C. for 3 h, counting the viable count in samples in 0 h and 3 h by a pouring plate method, culturing the poured plate at 37° C. for 48 h, measuring a survival rate, in which the survival rate is calculated as follows:

$$Survival rate / \% = \frac{{N}_{t}}{{N}_{0}} \times 100 ,$$

where No is the number of the viable count (CFU/mL) of the Lactobacillus helveticus ZJUIDS12 for 0 h; Nₜ is the number of the viable count (CFU/mL) of the Lactobacillus helveticus ZJUIDS12 for 3 h.

2. Bile Salt Resistance Experiment

Inoculating 1% activated and expanded Lactobacillus helveticus ZJUIDS12 suspension into the MRS liquid culture medium, culturing at 37° C. for 18 h, evenly mixing by the vortex, correcting the initial viable count to about 10⁹ CFU/mL, inoculating 10% the activated and expanded Lactobacillus helveticus ZJUIDS12 suspension into the MRS liquid culture medium containing 0.3% (m/v) ox bile salt (the MRS liquid culture medium without containing the ox bile salt is used as a control), culturing at 37° C. for 3 h, counting the viable count in samples by the pouring plate method, and culturing the poured plate at 37° C. for 48 h.

The bile salt resistance of the activated and expanded Lactobacillus helveticus ZJUIDS12 is expressed as a logarithm of a difference between the viable count in the 1 mL MRS liquid culture medium containing the bile salt and the viable count in the 1 mL MRS liquid culture medium without containing the bile salt at 3 h (log CFU/mL).

The above acid resistance and bile salt resistance are detected with the Lactobacillus rhamnosus ATCC53103 as the control.

As shown in table 6, the acid resistance and bile salt resistance of Lactobacillus helveticus ZUIDS12 are obviously better than that of the Lactobacillus rhamnosus ATCC53103. The survival rate of the Lactobacillus helveticus ZUIDS12 in the MRS medium with pH 3.0 is as high as 101.51%, and the viable count of the Lactobacillus helveticus ZUIDS12 in an environment containing the 0.3% bile salt still reaches more than 4*10⁶ CFU/mL, which indicates that the Lactobacillus helveticus ZUIDS12 has the good bile salt resistance, and thus has a strong viability in gastrointestinal tract.

TABLE 6
Results of the acid resistance and bile salt resistance of the strains

| | Acid resistance | Bile salt resistance |
|---|---|---|
| Strain | (%) | (ΔLog CFU/mL) |
| Lactobacillus helveticus ZJUIDS12 | 116.51 ± 0.41 | 0.47 ± 0.15 |
| Lactobacillus rhamnosus | 62.12 ± 0.18 | 1.03 ± 0.23 |
| ATCC53103 | | |

Probiotics needs to tolerate a series of adverse environments such as gastric acid and bile in the gastrointestinal tract and survive in order to play a probiotic role.

The Lactobacillus helveticus ZJUIDS12 provided by the application may grow and proliferate in an environment with pH 3.0, and thus may smoothly reach small intestine through the stomach. In addition, the Lactobacillus helveticus ZJUIDS12 may tolerate bile salt and survive in the intestinal tract, further thereby effectively improving the intestinal flora and playing a role in lowering the blood sugar level.

### Embodiment 8 Detection of Hydrophobicity of the Lactobacillus helveticus ZJUIDS12

1. Detection of the Hydrophobicity

Washing Lactobacillus precipitate with the 0.1 mol/L clean PBS buffer solution with pH 6.8 twice, then resuspending to make the absorbance value of OD610 about 0.5, and finally, obtaining the Lactobacillus suspension, in which the culture medium suspension is prepared in the same way as above;

mixing 2 mL of the Lactobacillus suspension with 2 mL of xylene thoroughly, shaking in water bath at 37° C. for 5 min, and measuring the absorbance value of OD610 of water phase after 0 h and 2 h, respectively.

$$Hydrophobicity ( \% ) = \frac{A 0 - At}{A 0} \times 100 ,$$

where A₀ is the absorbance value at 0 h, and Aₜ is the absorbance value at t h.

Results are shown in Table 7.

TABLE 7
Surface hydrophobicity of different strains (%)

| Strain | Hydrophobicity |
|---|---|
| Lactobacillus helveticus ZJUIDS12 | 25.87 ± 0.51% |
| Lactobacillus rhamnosus ATCC53103 | 12.31 ± 1.15% |

2. Result Analysis

The results show that the hydrophobicity of the Lactobacillus helveticus ZJUIDS12 is 25.87%, which is significantly higher than that of a reference standard strain. Therefore, it indicates that the Lactobacillus helveticus ZJUIDS12 has strong adhesive ability, which may adhere to human intestinal tract and further regulate the intestinal flora.

### Embodiment 9 Detection of Antibiotic Susceptibility of the Lactobacillus helveticus ZJUIDS12

Adding 1% of the about 10⁷ CFU/mL suspension of the Lactobacillus helveticus ZJUIDS12 cultured for 18 h to the sterilized MRS agar medium cooled to about 45° C., evenly mixing, and quantitatively adding to the petri dishes according to 15 mL per petri dish; after coagulation, taking a piece of antibiotic susceptibility paper with the tweezer, putting the antibiotic susceptibility paper on the sterilized MRS agar medium, putting the petri dishes face up in an incubator at 37° C. for 24 h, using a piece of paper without the antibiotics as the blank control, and measuring the diameter of each inhibition zone for 3 times.

The diameters of the inhibition zones of the Lactobacillus helveticus ZJUIDS12 to the antibiotic susceptibility is shown in table 8. According to the Antimicrobial Susceptibility Testing Clinical and Laboratory Standards Institute (CLSI) 2017, the Lactobacillus helveticus ZJUIDS12 is sensitive to penicillin G, ampicillin, cefazolin, amikacin, erythromycin, norfloxacin, and chloramphenicol, intermediate to gentamicin, and insensitive to ciprofloxacin and Sulfamethoxazole. Therefore, it may be concluded that the Lactobacillus helveticus ZJUIDS12 is sensitive to common antibiotics.

TABLE 8
Results for antibiotics sensitivity of the Lactobacillus helveticus
ZJUIDS12

| | | Diameter of | |
|---|---|---|---|
| | | inhibition zone | Susceptive |
| Name | Paper contents | (mm) | type |
| Penicillin G(P) | 10 U | 17.5 ± 0.12 | S |
| Ampicillin (AM) | 10 μg | 23.5 ± 0.05 | S |
| Cefazolin(CZ) | 30 μg | 14.5 ± 0.03 | S |
| Amikacin (AK) | 30 μg | 15.0 ± 0.05 | S |
| Gentamicin (GM) | 10 μg | 5.0 ± 0.02 | I |
| Erythromycin (E) | 15 μg | 17.5 ± 0.01 | S |
| Norfloxacin (NOR) | 10 μg | 13.0 ± 0.01 | S |
| Ciprofloxacin (CIP) | 5 μg | 0 | R |
| Sulfamethoxazole (SXT) | 1.25/23.75 μg | 0 | R |
| Chloramphenicol (C) | 30 μg | 15.5 ± 0.01 | S |

Note:
S: sensitive; I: intermediate; R: insensitive

With a wide application of the antibiotics in clinical treatment, the Lactobacillus becomes more and more insensitive. Long-term intake of the insensitive Lactobacillus brings great difficulties to the clinical treatment. However, the Lactobacillus helveticus ZJUIDS12 provided by the application is sensitive to the common antibiotics and not causes harm to human health.

### Embodiment 10 Detection of the Inhibition Ability of the Lactobacillus helveticus ZJUIDS12 to Pathogens

The inhibition activity of the Lactobacillus is detected by the international agar diffusion method: pouring 10 mL of a lysogeny broth (LB) agar culture medium into the sterile petri dishes as a lower culture medium after cooling, adding 1% of about 10⁷ CFU/mL indicator bacteria suspension after culturing for 18 h to the sterile LB agar culture medium cooled to about 45° C., evenly mixing, and quantitatively adding to the petri dishes according to 10 mL per petri dish; putting the sterile Oxford cups on the petri dishes, and gently pulling out the Oxford cups after an upper culture medium is condensed; quantitatively adding the fermentation supernatant of the Lactobacillus helveticus ZJUIDS12 at 100 μL/well, and selecting strains with the obvious inhibition zones around the wells by using the 0.1 mol/L PBS buffer solution with pH 6.8 as the control, and measuring the diameter of each inhibition zone for 3 times.

As shown in table 9, metabolites of the Lactobacillus helveticus ZJUIDS12 have certain inhibitory effects on Staphylococcus aureus, Escherichia coli, Salmonella enteritidis, Listeria monocytogenes and other pathogens, which is better than that of ATCC53103. Therefore, the metabolites of the Lactobacillus helveticus ZJUIDS12 have inhibition ability to the pathogens.

TABLE 9
Test results of the inhibition ability of the strains to the pathogens

| | Diameter of inhibition zone (mm) | | | |
|---|---|---|---|---|
| | Staphylo- | | Salmonella | Listeria mono- |
| | coccus | Escherichia | enteritidis | cytogenes |
| Strain | aureu S4050 | coli JM101 | ATCC50335 | CMCC 26003 |
| Lactobacillus | 15.5 ± 0.05 | 15.0 ± 0.01 | 19.5 ± 0.03* | 21.5 ± 0.02* |
| helveticus | | | | |
| ZJUIDS12 | | | | |
| Lactobacillus | 14.5 ± 0.02 | 16.5 ± 0.05 | 16.2 ± 0.05 | 15.2 ± 0.05 |
| rhamnosus | | | | |
| ATCC53103 | | | | |

The Staphylococcus aureus is the most common pathogen causing human suppurative infection, some Escherichia coli may cause severe diarrhea and septicemia, and some Salmonella species may also cause human food poisoning. However, the metabolites of the Lactobacillus, such as bacteriocins, organic acids, the hydrogen peroxide and other antibacterial substances, may inhibit growth of the pathogens individually or jointly. The metabolites of the Lactobacillus helveticus ZJUIDS12 provided by the application has certain antagonistic effects on these three pathogens, which plays an important role in maintaining intestinal microecological balance and promoting good health.

### Embodiment 11 Preparation of Functional Fermented Yogurt by the Lactobacillus helveticus ZJUIDS12

1. Processing Flow of the Yogurt

Raw materials→Preheating→Homogenizing→blending→Sterilization→Cooling→Preparation and inoculation→Fermentation→After-ripening→Refrigeration

2. Key Points of Operation

(1) Raw materials: 2 L of ultra-high temperature (UHT) sterilized whole milk or fresh whole milk;

(2) Preheating: heating to 63° C. in a container;

(3) Homogenizing: pouring into a homogenizer to homogenize at a pressure of 15-25 MPa, pouring mixed solution into an iron can, adding 100 g of white sugar, and sterilizing in a water bath at 90° C. for 10 min;

(4) Blending: adding ingredients into the milk and dissolving;

(5) Sterilization: sterilizing the milk added with the white sugar in the water bath at 90° C. for 10 min;

(6) Cooling: cooling the sterilized milk to 40-50° C. for later use;

(7) Preparation of starter: inoculating strains of the Lactobacillus helveticus ZJUIDS12 in a test tube with sterilized skim milk (12%, w/v) in a sterile environment, and culturing at 37° C. for 20 h, in which an inoculation amount is 2-4% (v/v) for each passage, and passaging the strains for 2-3 times to restore vitality, and storing the test tube in the refrigerator at 4° C.;

(8) Inoculation and fermentation: inoculating the activated Lactobacillus helveticus ZJUIDS12 with the inoculation amount of 2-4% (v/v) under aseptic condition, and fermenting the yogurt at 42° C. for 6-10 h;

(9) After-ripening: after the fermentation, putting the fermented yogurt into the refrigerator at 4° C. for after-ripening for 12-24 h; and

(10) Filling and refrigerating: after the after-ripening, filling the fermented yogurt into 250 mL of sterile glass bottles and refrigerating in a freezer.

### Embodiment 12 Preparation of Functional Fermented Fruit and Vegetable Juice by the Lactobacillus helveticus ZJUIDS12

1. Processing Flow of the Fermented Fruit and Vegetable Juice

Raw materials→Cleaning→Flash evaporation→Mashing→Blending→Homogenizing→Sterilization→Cooling→Inoculating→Closed fermentation→After-ripening→Filling→Refrigerating

2. Key Points of Operation

(1) Raw materials: selecting fresh Cucurbita moschata and Hylocereus undatus;

(2) Cleaning and cutting into pieces: cleaning the Cucurbita moschata and Hylocereus undatus, peeling (removing pulp from the Cucurbita moschata) and cutting into small pieces;

(3) Flash evaporation: inactivating enzymes by the flash evaporation at 121° C. for 0.5-1 min, and quickly exhausting;

(4) Mashing: according to a weight ratio of pumpkin:water=1:1, gradually putting the Cucurbita moschata and the water into a colloid mill for grinding, in which coarse grinding and fine grinding are respectively carried out once, and mashing the Hylocereus undatus by a pulping machine until fruit pulp is uniform and has no blocks;

(5) Blending and homogenizing: according to 15% of Cucurbita moschata juice and 30% of Hylocereus undatus juice, adjusting a soluble solid content to 10° Brix with sucrose, adding 0.2% stabilizer CMC, evenly mixing, using a two-stage homogenization method, in which a low pressure of 15 MPa is firstly carried out, and then a high pressure of 25 MPa is carried out, so that a particle diameter of melon pulp is 2-3 μm;

(6) Sterilization and cooling: keeping a temperature of the blended compound fruit and vegetable juice at 100° C. for 10 min and cooling to about 40° C.;

(7) Inoculation and fermentation: inoculating the activated Lactobacillus helveticus ZJUIDS12 under the aseptic condition, controlling the initial bacterial count at 10⁷ CFU/mL, and fermenting at 37° C. for 24 h;

(9) After-ripening: after the fermentation, putting the fruit and vegetable juice into the refrigerator at 4° C. for after-ripening for 3 h; and

(10) Filling and refrigerating: after the after-ripening, filling the fruit and vegetable juice into 250 mL of the sterilized glass bottles and refrigerating in the freezer.

### Embodiment 13 Preparation of the Lactobacillus helveticus ZJUIDS12 Powder

1. Preparation of the Lactobacillus helveticus ZJUIDS12 Sludge

Selecting the single colonies of the Lactobacillus helveticus ZJUIDS12, inoculating into 50 mL of the MRS liquid culture medium, culturing in an incubator at 37° C. for 18 h, activating in 250 mL of the MRS liquid culture medium according to 5% of a inoculum size, culturing in the incubator at 37° C. for 24 h, then culturing the activated Lactobacillus helveticus ZJUIDS12 in a 10 L fermenter with the 5% inoculum size for high-density anaerobic culture at 37° C. and pH 6.8 for 18 h, centrifuging at 8000 r/min for 15 min at 4° C., discarding the supernatant, collecting the Lactobacillus helveticus ZJUIDS12 precipitate, and rinsing the Lactobacillus helveticus ZJUIDS12 with the sterile phosphate buffer solution with pH 7.0 for 2 times to obtain the Lactobacillus helveticus ZJUIDS12 sludge.

2. Preparation of Protective Agent

Freeze-drying protective agent contains 15% of skim milk powder, 5% of trehalose, 3% of sodium glutamate, 1% of the glycerol and 0.5% of cysteine hydrochloride. Water is used as solvent and sterilized at 110° C. for later use.

3. Preparation of the Lactobacillus helveticus ZJUIDS12 Powder

Fully and evenly mixing the prepared Lactobacillus helveticus ZJUIDS12 precipitate with a protective agent solution according to the ratio of 1:5, pre-freezing above mixture at −40° C. for 5 h to evenly freeze the mixture on inner wall of a container, then carrying out vacuum freeze drying for 18-20 h to obtain the Lactobacillus helveticus ZJUIDS12 powder, and rehydrating the Lactobacillus helveticus ZJUIDS12 powder with SPSS and washing twice,

The number of the viable count detected in the Lactobacillus helveticus ZJUIDS12 powder is about 1.0×10¹¹-1×10¹² CFU/g.

### Embodiment 14 Preparation of Probiotic Milk Powder for Pets by the Lactobacillus helveticus ZJUIDS12

1. Preparation of the Lactobacillus helveticus ZJUIDS12 Powder

Lactobacillus helveticus ZJUIDS12 lyophilized powder is prepared according to embodiment 13, and the viable count of the lyophilized powder is about 1.0×10¹¹-1×10¹² CFU/g.

2. Preparation of the Formula Milk Powder for Pets

Raw materials: the milk powder, fish meal, bone meal, grains and vegetable oil;

Additives: vitamins, trace elements, functional factors, and others;

Automatic batching: putting above raw materials into a material bin according to a formula;

Crushing: crushing the weighed materials by a crusher;

Mixing: adding the vegetable oil and the trace elements into the crushed materials, and putting mixed materials into a mixer for evenly mixing;

Puffing: making the mixed materials into granular materials by a bulking machine;

Drying: drying the mixed materials by a dryer at 65-70° C.;

Classification screening: passing the mixed materials flow through a classification screen, and controlling the particle diameter to be 2.5-5 mm

3. Preparation of Probiotic Formula Powder for Pets

The Lactobacillus helveticus ZJUIDS12 powder and the formula milk powder for pets are mixed according to the ratio of 1:100, so that the viable count in final products is more than 10⁸ CFU/g, then the final products are filled and sold.

### Embodiment 15 Preparation of Postbiotics by the Lactobacillus helveticus ZJUIDS12

1. Preparation of the Lactobacillus helveticus ZJUIDS12 Sludge

Selecting the single colonies of the Lactobacillus helveticus ZJUIDS12, inoculating into 50 mL of the MRS liquid culture medium, culturing in the incubator at 37° C. for 18 h, activating in 250 mL of the MRS liquid culture medium according to 5% of the inoculum size, culturing in the incubator at 37° C. for 24 h, then culturing the activated Lactobacillus helveticus ZJUIDS12 in the 10 L fermenter with the 5% inoculum size for the high-density anaerobic culture at 37° C. and pH 6.8 for 18 h, centrifuging at 8000 r/min and 4° C. for 15 min, discarding the supernatant, collecting the Lactobacillus helveticus ZJUIDS12 precipitate, and rinsing the Lactobacillus helveticus ZJUIDS12 with the sterile phosphate buffer solution with pH 7.0 for 2 times to obtain the Lactobacillus helveticus ZJUIDS12 sludge.

2. Preparation of the Postbiotics of the Lactobacillus helveticus ZJUIDS12

Heat-killing the collected Lactobacillus helveticus ZJUIDS12 sludge, Lactobacillus helveticus ZJUIDS12 or fermentation broth under the high pressure at 121° C. for 15 min, collecting heat-killed Lactobacillus helveticus ZJUIDS12 sludge, the heat-killed Lactobacillus helveticus ZJUIDS12 or the fermentation broth, and directly using or adding the heat-killed Lactobacillus helveticus ZJUIDS12 sludge, heat-killed Lactobacillus helveticus ZJUIDS12 or fermentation broth into other products after vacuum packaging or aseptic filling.

It should be understood that the technical schemes of the present application are not limited to the limits of the above specific embodiments, and any technical variations made according to the technical schemes of the present application, without departing from the scope protected by the objective and claims of the present application, fall within the scope of protection of the present application.

What is claimed is:

1. Lactobacillus helveticus ZJUIDS12 with a strain preservation number of CGMCC NO. 23997.

2. An application of the Lactobacillus helveticus ZJUIDS12 according to claim 1 in preparing products for treating liver injury.

3. The application according to claim 2, wherein the products for treating the liver injury comprise Lactobacillus helveticus ZJUIDS12 powder preparations, heat-killed Lactobacillus helveticus ZJUIDS12 or fermentation broth products.


SEQUENCE LISTING (1 sequence)

SEQ ID NO: 1 - DNA, 1493 bp, synthetic construct
  source 1..1493: mol_type = other DNA
caggacgaacgctggcggcgtgcctaatacatgcaagtcgagcgagcagaaccagcagatttacttcggtaatgacgctggggacgcgagcggcggatgggtgagtaacacgtggggaacctgccccatagtctgggataccacttggaaacaggtgctaataccggataagaaagcagatcgcatgatcagcttataaaaggcggcgtaagctgtcgctatgggatggccccgcggtgcattagctagttggtaaggtaacggcttaccaaggcaatgatgcatagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggatagaggcagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactccatgtgtagcggtggaatgcgtagatatatggaagaacaccagtggcgaaggcgactctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttaaaactcaaaggaattgacgggggcccgcacaagcggtggagcatgtggtttaattcgaagcaacgcgaagaaccttaccaggtcttgacatctagtgccatcctaagagattaggagttcccttcggggacgctaagacaggtggtgcatggctgtcgtcagctcgtgtcgtgagatgttgggttaagtcccgcaacgagcgcaacccttgttattagttgccagcattaagttgggcactctaatgagactgccggtgacaaaccggaggaaggtggggatgacgtcaagtcatcatgccccttatgacctgggctacacacgtgctacaatggacagtacaacgagaagcgagcctgcgaaggcaagcgaatctctgaaagctgttctcagttcggactgcagtctgcaactcgactgcacgaagctggaatcgctagtaatcgcggatcagaacgccgcggtgaatacgttcccgggccttgtacacaccgcccgtcacaccatggaagtctgcaatgcccaaagccggtggcctaaccttcgggaaggagccgtctaaggcagggcagatgactggggtgaagt